(19) United States
(12) Plant Patent Application Publication
(10) Pub. No.: US 2006/0179526 P1
(43) Pub. Date: Aug. 10, 2006

(54) Mimosa plant 'Ramos'

(50) MIMOSA SENSITIVA; MIMOSA

(76) Inventor: Guillermo Ramos Gonzales, Texcoco (MX)

Correspondence Address: ERIC HANSCOM, 7395 PORTAGE WAY, CARLSBAD, CA, 92011, US

(21) Appl. No.: 11/038,681

(22) Filed: Jan. 21, 2005

(51) Int. Cl. A01H 5/00

(52) U.S. Cl. PLT/226

(57) ABSTRACT

A new Mimosa or sensitive plant which has shorter growth habit, reduction in the development of the vegetative parts, more resistance to lower temperatures and cold winds, and requires less water for survival and growth. The variety successfully propagates from layering and plant division, and is suitable for year round production in outdoor environments up to 3,500 feet altitude, outside of its traditional geographic range, as a flowering pot plant or an ornamental shrub. There are also potential uses of this plant for military operations and home security, as well as medicinal and general amusement purposes. This new and distinct variety has shown to be uniform and stable in the resulting generations from asexual propagation.

### CROSS REFERENCE TO RELATED APPLICATIONS

None.

### STATEMENT REGARDING FEDERALLY SPONSORED RESEARCH OR DEVELOPMENT

This invention was not federally sponsored.

### BACKGROUND OF THE INVENTION

### Field of the Invention

The plant Mimosa sensitive, or sensitive plant, was first described in Brazil but is now a pan-tropical weed with healthy populations in Mexico, Latin America, South America, Asia, Australia, and Oceania. It is called sensitive plant because is has pulvini which, when touched or subjected to extreme wind, light, or temperature variation, cause the leaf to fold up and droop, making it appear substantially less attractive to grazing animals; thus it has considerable novelty value to humans who like to watch it close up upon touching. Once the stimulus has passed, the leaves recover their natural position and arrangement. There have been numerous attempts to introduce Mimosa sensitiva to regions outside of its native tropical, humid environment, but until this invention the plant has been very difficult to grow in temperate zones.

The present invention constitutes a new and distinct variety of Mimosa or sensitive plant, which was developed by gathering naturally occurring plants in the Northwest of the state of Veracruz, Mexico, shortly after January in the year 1999, at an altitude of approximately 1,000 meters (3,280 feet) above sea level, and a warm humid climate with average temperatures of 23 degrees Celsius (73.4 degrees Fahrenheit), and an annual precipitation of 1600-1800 mm (6-7 inches). These plants were then transported to Texcoco, a town located to the east of the State of Mexico, in Mexico, which is located at an altitude of 2,500 meters (8,200 feet) and has a climate characterized by a semidry climate with freezing temperatures in the winter, an average temperature of 15.9 degrees Celsius (60.6 degrees Fahrenheit), and an average annual precipitation of 686 mm (2.0 inches). Once transported, the plants were first cultivated in a greenhouse under similar conditions to that of their place of origin for a period of six months. Following this, the plants were cultivated freely for three months outside of the greenhouse under natural ambient weather conditions and daily watering, with regular fertilizer and maintenance. At the end of three months, the plants were watered every third day, with regular fertilizer and maintenance. At the end of the first year, 25 percent of the plants survived, and were allowed to cross pollinate, and the seeds were sown. From the end of the first year, watering has been decreased to only twice a week with less water during the rainy season. Cross pollination and selection of plants through survival of the sometimes freezing temperatures during the winter have eventually produced a new variety which can be propagated through layering and plant division. The new Mimosa, or sensitive plant, ‘Ramos’, may be distinguished from its ancestral parent plant, an unnamed seedling, by the following combination of characteristics:
1. The unnamed seeding has a breeding background in unnamed seedlings.
2. ‘Ramos’ has shorter growth habit of approximately 45 cm (15.7 inches) maximum, while the unnamed seedling may reach maximum heights of 122 cm (48 inches).
3. ‘Ramos’ has a reduction in the development of the vegetative parts relative to the unnamed seedling.
4. ‘Ramos’ has more resistance to lower temperatures than the unnamed seedling.
5. ‘Ramos’ has more resistance to cold winds than the unnamed seedling.
6. ‘Ramos’ has the ability to survive and grow on less water than the unnamed seedling.

### BRIEF SUMMARY OF THE INVENTION

Initial asexual propagation of ‘Ramos’ by layering and plant division was first done in Texcoco, Mexico. These two forms of reproduction were done in a greenhouse under controlled conditions.

‘Ramos’ has proven to be stable by propagation with layering and plant division in several generations. ‘Ramos’ is a low, stunted sensitive plant with high vigor.

The object of the selection of this sensitive plant variety was to create a sensitive plant with:
1. Greater resistance to lower temperatures than its parent plants.
2. Greater resistance to cold winds than its parent plants.
3. Greater ability to survive and grow on less water than its parent plants.
4. Greater ability to grow outside of the tropical, humid environment from which it originated.
5. Suitability for growth in temperate environments as an indoor or outdoor plant.
6. Suitability for growth in frigid zones as an indoor plant.
7. Suitability for growth in desert zones as an indoor plant.

This combination of qualities was not present in previously available commercial or naturally occurring cultivars of this type and distinguish ‘Ramos’ from all other varieties of which we are aware.

The seeds from ‘Ramos’ along with layerings and plant divisions were planted in both controlled environments and outdoors, and evaluations were conducted on the resulting plants. ‘Ramos’ was selected by inventor Guillermo Ramos Gonzalez, in his plant developmental program in Texcoco, Mexico.

### BRIEF DESCRIPTION OF THE DRAWINGS

The accompanying color illustrations show as true as is reasonably obtainable in color photographs of this type the typical characteristics of the leaves, stems, and growth habit of ‘Ramos’, specifically illustrated in:

FIG. 1. Open leaves.

FIG. 2. Closed leaves.

FIG. 3. Full plant.

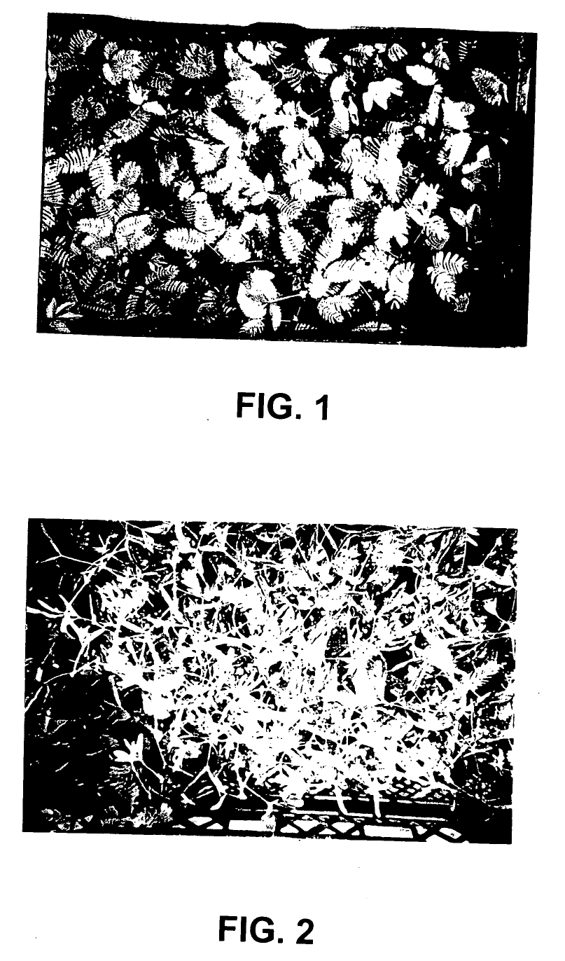

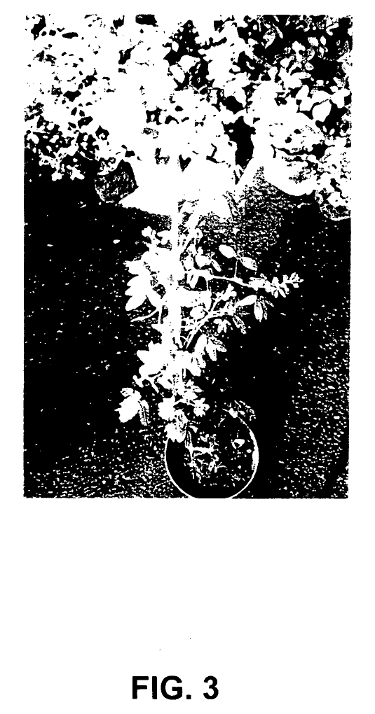

### DETAILED BOTANICAL DESCRIPTION OF THE VARIETY

The following is a detailed description of the sensitive plant: Mimosa sensitiva var. ‘Ramos’.
Kingdom: Plantae
Phyla: Magnoliophyta
Class: Mangoliopsida
Subclass: Rosidae
Order: Fabaceae
Family: Mimosaceae
Genus: Mimosa
Species: sensitiva The following observations, measurements, values and comparisons describe plants grown in outdoor conditions in Texcoco, Mexico. The age of the observed plants was approximately three months after propagation by cuttings, in the case of the plants photographed, and produced as pot plants in containers of approximately 13 cm. in diameter. There are no color references given as color is not a distinguishing characteristic of this new plant.
Parents: unnamed seedling.
Times: Unnamed seedling.
Classification:
Botanical: sensitiva variety.
Commercial: Variety.
Plant:
   Plant growth.—Moderately vigorous. Grows compact upright, perennial plant, herbaceous-branched type, with thorns, on the base and all along the shaft, has bipinaded leaves formed by follicles. Fibrous root type, which develops an average depth of 40 cm (15.7 in). Plant has inflorescence of pink to light purple color, clumping together. Plants will reach average maximum height of 45 cm (15.7 inches) in approximately 4-6 months. Roots will form from layering in approximately 6 weeks.
Stems: Reddish-brown, prickly.
   Stem diameter.—2-4 mm.
   Internode length.—2-4 cm.
Plant foliage: Bipinnate, finely divided, fern-like, pale green leaves formed by follicles, closing when disturbed by pulvini, pinnae 4, often reddish.
   Quantity of leaves.—12-25 pairs per lateral branch, acute, bristly.
   Leave size.—Small, 1-3 cm long times 1-2 cm wide.
Inflorescence:
   Blooming habit.—Twice per year.
   Number of flowers.—Generally 3 buds per plant. Usually in rainy season.
   Flower shape.—Spherical, globose heads, radial symmetry, nearly 1 cm in diameter. Typical flowering head with stamens dominating as the attractive components of the flowers. Flowers are auxiliary and have peduncle up to 2.5 cm long.
   Flower color.—Pink to pale lilac.
   Duration.—As a pot plant or in ground, flowers last 15-40 days.
   Fragrance.—None.
Pods. Crowded, flat, prickly-bristly, indented between the few (2-4) seeds, to nearly 1 cm long.
Propagation: Bristles on seed pods cling to fur and clothing, about 2 mm broad, rounded, brown. Winter hardiness, wind hardiness, drought tolerance. This plant has been specifically developed to be winter hardy, wind hardy, and drought tolerant, and has been tested for the same.

1. A new and distinct variety of Mimosa plant, substantially as herein illustrated and described as a distinct and novel Mimosa variety due to its shorter growth habit, reduction in the development of the vegetative parts, more resistance to lower temperatures and cold winds, and ability to survive and grow on less water for than the parent plant, suitability for propagation from layering and plant division, its suitability for year round production in outdoor environments up to 3,500 feet altitude, outside of its traditional geographic range, as a flowering pot plant or an ornamental shrub, and its potential uses for military uses and home security, as well as general amusement purposes and medicinal uses, which make the variety suitable for distribution in the landscaping and other industries.
